(19) United States
(12) Patent Application Publication
(10) Pub. No.: US 2004/0101499 A1
(43) Pub. Date: May 27, 2004

(54) Multi-sensory pleasant lip gloss

(76) Inventor: Jillian Jacobson-Alti, Coral Gables, FL (US)

Correspondence Address: RUDEN, MCCLOSKY, SMITH, SCHUSTER & RUSSELL, P.A., P.O. BOX 1900, FORT LAUDERDALE, FL, 33301, US

(21) Appl. No.: 10/718,213

(22) Filed: Nov. 20, 2003

Related U.S. Application Data
(60) Provisional application No. 60/428,007, filed on Nov. 21, 2002.

(51) Int. Cl. A61K 7/025

(52) U.S. Cl. 424/64

(57) ABSTRACT

An improved stable lip gloss composition consisting essentially of at least one lower alkyl ester of a fatty acid having 12 to 18 carbon atoms that is liquid at 25° C., an effective bodying amount of at least one organic bodying agent having a solubility in the fatty ester of at least 1 percent by weight at 25° C., an amount effective in imparting a pleasant odor of at least one odorant, an amount effective in imparting a pleasant taste of at least one flavorant, and an effective amount of at least one colorant

### CROSS REFERENCE TO RELATED APPLICATION

This application claims the benefit of U.S. Provisional Application Serial No. 60/428,007, filed Nov. 21, 2002, entitled MULTI-SENSORY PLEASANT LIP GLOSS.

### BACKGROUND OF THE INVENTION

### Field of the Invention

This invention relates to an improved lip gloss composition capable of providing visual pleasure to the user and to observers, as well as providing gustatory, olfactory, and tactile pleasure to the user, and to a method of providing visual, gustatory, olfactory, and tactile pleasure to a person by applying such a composition to the person's lips.

The application of color to a person's lips in order to increase the visual contrast between the lips and the adjacent facial skin has been known since ancient times. The range of colors used for the purpose has expanded beyond traditional pink, red, beige and brown shades to nearly the entire spectral range. Incorporation of scents into lip gloss compositions has traditionally been carried out to mask harsh or irritating odors of certain ingredients in these compositions. The present inventors have not found a record of deliberate addition of flavor or other taste modifying agent to colored lip gloss.

To be acceptable to users, a lip gloss composition must have a consistency and degree of uniformity that permits easy application to the user's lips, and maintain that consistency and uniformity substantially without objectionable change in properties such as phase separation, thickening, or color change during a possibly extensive period of time in commercial channels before being purchased by the user and a further period while being gradually used up. For convenience such a composition is termed stable.

### SUMMARY OF THE INVENTION

It is therefore an object of the invention to provide a stable lip gloss composition that overcomes the deficiencies of compositions of this type available at the time of this application, and that pleases the user's senses of sight, taste, smell, and touch while pleasing onlookers' sense of sight also by its intense color.

In accordance with this invention, therefore, there is provided a stable lip gloss composition consisting essentially of at least one lower alkyl ester of a fatty acid having 12 to 18 carbon atoms that is liquid at 25° C., an effective bodying amount of at least one organic bodying agent having a solubility in the fatty ester of at least 1 percent by weight at 25° C., an amount effective in imparting a pleasant odor of at least one odorant, an amount effective in imparting a pleasant taste of at least one flavorant, and an amount of at least one colorant imparting an intense color to the composition.

The term “stable” is used to indicate that the composition retains its physical integrity and therapeutic effectiveness for a minimum of six months when kept at a temperature in the range of 5° C. to 40° C. including different temperatures within that range.

The term “consisting essentially of” is used in its art-recognized meaning to indicate that the composition is open to the inclusion of unstated other ingredients only to the extent that such other ingredients do not materially affect the desirable and beneficial properties of the defined composition. Major amounts of water, for example, are excluded as likely to cause phase separation and other manifestations of instability in the composition of the invention, while small amounts of water as may be present as a result of exposure to humidity can be tolerated.

The term “intense color” is used to indicate that, unlike previously available flavored lip gloss compositions, the full-bodied lipstick-like color of the composition as supplied maintains its intensity when applied to the user's skin, for example when rubbed on the user's hand. Previously available flavored lip gloss compositions lose color when tested in this manner.

The term “lower alkyl” is used to refer to alkyl groups having one to four carbon atoms.

The consistency of the composition of the invention is controlled to be sufficiently flowable for ease in packaging and removing from a container, ease in application to the user's lips while minimizing the tendency to smudge or migrate from the lips to other areas of the user's skin. The composition can be thixotropic.

### DESCRIPTION OF PREFERRED EMBODIMENTS

In the composition of the invention, the concentration of lower alkyl ester is in the range of 10-80 grams per 100 grams of the composition, preferably 25-65 grams per 100 grams; the concentration of bodying agent is in the range of 3-60 grams per 100 grams of the composition, preferably 15-35 grams per 100 grams; the concentration of odorant is in the range of 0.0002-2 grams per 100 grams of the composition, preferably 0.0005-0.5 grams per 100 grams; the concentration of flavorant is in the range of 0.0002-2 grams per 100 grams of the composition, preferably 0.0005-0.5 grams per 100 grams; and the concentration of colorant is in the range of 0.0002-2 grams per 100 grams of the composition, preferably 0.001-1 grams per 100 grams.

The lower alkyl fatty acid ester, bodying agent, odorant and flavorant ingredients of the composition can be combined with little or no other material present into a concentrate suitable for facilitating the subsequent compounding with colorant to provide a variety of formulations presenting the composition of this invention. Such concentrates can conveniently include 10-80 parts by weight of lower alkyl fatty acid ester, 3-60 parts by weight of bodying agent, 0.0005-3 parts by weight of odorant, and 0.0005-3 parts by weight of flavorant

The lower alkyl ester of fatty acid having 12 to 18 carbon atoms according to the invention can be, for example, a methyl, ethyl, n-propyl, isopropyl, n-butyl, sec-butyl and isobutyl ester of isostearic, lauric, linoleic, myristic, oleic, palmitic, ricinoleic and stearic acid and mixtures thereof. Ester mixtures resulting from the alcoholysis of natural triglyceride fats and oils and the esterification of commercially available fatty acid mixtures are particularly suitable. Preferred esters include methyl esters of soybean oil fatty acids (so-called methyl soyate), methyl oleate, butyl stearate, and ethyl esters of coconut fatty acids. Isopropyl myristate and isopropyl palmitate are particularly preferred.

The organic bodying agent present in the composition of the invention in an amount effective to adjust the consistency and viscosity of the composition when dissolved therein can be a crystalline solid, a waxy solid, an amorphous solid, or a viscous liquid. Chemically the bodying agent can be a long chain aliphatic amide, ester, hydrocarbon, or ketone having at least 30 carbon atoms and mixtures thereof, illustrated by stearamide, ethylenebis (stearamide), hydrogenated castor oil, butylenes glycol ester of montan wax acids, hydrogenated tallow mixed monoglycerides and diglycerides, ozokerite mineral wax, petrolatum, polybutene (viscous liquid resulting from catalytic polymerization of an isobutylene-rich butane stream) and di-n-heptadecyl ketone. Ozokerite, petrolatum and polybutene are particularly preferred.

The colorant present in the composition of the invention contributes to the desired appearance according to prevailing esthetics at any given time as well as to the consistency of the composition. Colorants are available to impart any desired color as well as special effects such as iridescence, pearlescence, and shimmer. Preferred colorants are particulate solids with minimal solubility or no solubility in the liquid phase of the composition, and can include D&C Red #6 Barium Lake, D&C Red #7. Calcium Lake, FD&C Yellow #5 Aluminum Lake, Titnanium Dioxide, Iron Oxides, Mica, Bismuth Oxychloride, Silica, Carmine Red, Ferric Ferrocyanide and mixtures thereof. The colorant is preferably finely ground to a maximum particle size of 325 mesh before blending into the composition or predispersed in a portion of the liquid phase. Commercially available colorant dispersions can be used to advantage.

Dispersion of colorant in the liquid phase of the composition can be assisted by addition of small amounts of special components known as surfactants that inhibit or delay the separation of the phases. Useful surfactants can be anionic, cationic, nonionic, or zwitterionic. Many representatives of each type are known and commercially available. Particularly preferred surfactants include sodium, potassium, and triethanolamine salts of oleic and stearic acids (which can be prepared in situ by including in the formulation suitable sodium, potassium and amine bases along with the desired acids), dioctyl sodium sulfosuccinate, sodium dodecyl sulfate, glycerol monooleate, glycerol monostearate, sorbitan sesquioleate and ethoxylated sorbitan esters such as Polysorbate 20, Polysorbate 65 and Polysorbate 80. The amount of surfactant when used is a small fraction of the amount of liquid phase, preferably in the range from 0.1 grams to 10 grams per 100 grams of liquid phase.

The colorant, odorant, and flavorant ingredients of the composition of the invention can be selected independently according to the effect desired. Natural fruit flavors can be combined with the colors naturally associated with the particular fruit, such as grape flavor in a purple colored composition and cherry flavor in a red composition, but fanciful combinations of taste and color departing from the natural associations can be provided as desired.

Antioxidants and preservatives such as benzalkonium chloride, di-coco-dimethylammonium chloride, dilauryl thiodipropionate, methyl parahydroxybenzoate, propyl parahydroxybenzoate, and tocopherol can be included as needed. Such antioxidants and preservatives when present typically do not exceed 1% by weight of the composition and preferably occur within a range of 0.0001 to 0.3% by weight.

The odorant in the composition of this invention can be any odorant imparting perceptible and pleasant odor characteristic to the composition during its useful life, and can be natural or synthetic in origin. Suitable natural and synthetic odorant substances include those compiled by the US Food and Drug Administration in Title 21 of the Code of Federal Regulations, Sections 172.510 and 172.515 respectively, whether there classified for regulatory purposes as odorants or as flavorants. Particularly suitable odorants include basil, bergamot, cilantro, citrus, jasmine, lemongrass, menthol, musk, pine oil, rosemary, sage, sandalwood, thyme, and vanilla, and mixtures thereof. Sources of fruity or sweet odor are particularly preferred.

The flavorant in the composition of this invention can be any flavorant imparting perceptible and pleasant bitter, refreshing, sour, spicy or sweet flavor characteristic to the composition during its useful life, and can be natural or synthetic in origin. Suitable natural and synthetic flavorant substances include those compiled by the US Food and Drug Administration in Title 21 of the Code of Federal Regulations, Sections 172.510 and 172.515 respectively, whether there classified for regulatory purposes as odorants or as flavorants. An appropriate amount of a single substance can serve as flavorant, odorant, or both. Particularly suitable flavorants include almond, blueberry, cappuccino, cherry, chocolate, cinnamon, coconut, coffee, grape, orange, pineapple, tea, vanilla, watermelon, natural and artificial sweeteners, as well as extracts, concentrates, and mixtures thereof.

Oxidation inhibitor and/or ultraviolet absorber when present can be odorless and tasteless or possess an agreeable odor and/or taste. Suitable oxidation inhibitors include Vitamin C ascorbic acid, its salts and esters, and Vitamin E tocopherol as natural prototypes of the category, as well as the vitamin-inactive isomer erythorbic acid, oxy-acids of phosphorus such as phosphoric acid and polyphosphoric acid, aliphatic hydroxypolycarboxylic acids such as citric acid, malic acid, and tartaric acid, EDTA and its sodium and calcium salts, and alkyl-substituted phenols such as BHT, BHA, thymol, carvacrol, 4,4′-butylidenebis(2-t-butyl-5-methylphenol), 1,1,3-tris(2-methyl-4-hydroxy-5-t-butylphenyl)butane and 3,5-di-t-butyl-4-hydroxyphenylpropionic acid and its esters with C1-C18 monohydric alcohols or 2-6 functional polyhydric alcohols. Suitable ultraviolet absorbers absorb radiation in the range of wavelengths from about 270 nm to about 400 nm and include salicylic acid esters, 2-hydroxy-4-alkoxybenzophenones, and substituted derivatives of 2 (2′-hydroxy-5′-alkylphenyl) benzotriazole. When present, the proportion of oxidation inhibitor and/or ultraviolet absorber is generally in the range from 0.001% to 0.5 by weight, preferably from 0.005% to 0.1% by weight of the composition.

In addition to the essential fatty acid alkyl ester, bodying agent, odorant, flavorant, and colorant components, the composition of the invention can include d such adjuvants as are helpful for convenient preparation, stabilization, dispensing and application of the composition.

Compositions according to this invention can be prepared by conventional procedures. To minimize contamination from the growth of microorganisms, sterilized equipment is preferably used. Once blended, the composition can be packaged and stored in any suitable container inert to the contents including aluminum, glass, stainless steel, and solvent resistant plastics including polyamide, polycarbonate, polyester, polypropylene, and ABS polymer. Storage is preferably in a cool place away from strong light. Continued sterility can be assured by conventional techniques including aseptic packaging and post-sterilization in the final package by electron beam exposure.

In use, compositions according to this invention are applied to the user's lips in any suitable manner. To illustrate, the composition can be presented to the user in a cylindrical container whose diameter and length approximate the dimensions of a human finger and that is fitted with a closure carrying an applicator having a rod reaching nearly to the bottom of the container and terminating in a brush, a wand component or applicator, so as to deliver at a time enough composition to be brushed or painted on one of the user's lips.

### EXAMPLE A

### Preparation of Colorant Dispersions

Three grams of each colorant shown below are dispersed in a mixture of 2 grams sorbitan sesquioleate with 45 grams isopropyl palmitate by agitation at 30° C. until a smooth dispersion is obtained.

| Dispersion No. | Colorant |
|---|---|
| I | D & C Red #6 Barium Lake |
| II | D & C Red #7 Calcium Lake |
| III | F D & C Yellow #6 Aluminum Lake |
| IV | Titanium Dioxide |
| V | Red and Yellow Iron Oxides (3:1 Mixture) |
| VI | Mica |
| VII | Bismuth Oxychloride |
| VIII | Silica |
| IX | Carmine Red |
| X | Ferric Ferrocyanide |

### EXAMPLES 1-11

### Preparation of Stable Lip Gloss Compositions According to the Invention

Mixtures are prepared of isopropyl myristate and/or isopropyl palmitate lower alkyl fatty acid ester, petrolatum, polybutene, and/or ozokerite wax organic binder, flavorant and odorant and auxiliary ingredients as shown below, and are combined with one or more colorant dispersions of Example A above in the amounts shown, the ingredients identified as flavors serving both as flavorant and as odorant. All parts are by weight. Each resulting composition is packaged in clear glass tubes fitted with an applicator and holding about 25 grams each.

| Example | 1 | 2 | 3 | 4 | 5 | 6 | 7 | 8 | 9 |
|---|---|---|---|---|---|---|---|---|---|
| Isopropyl palmitate | 50 | — | 20 | 60 | 50 | — | 20 | 60 | 50 |
| Isopropyl myristate | — | 50 | 20 | — | — | 50 | 20 | — | — |
| Petrolatum | 22 | 7 | 10 | 7 | 22 | 7 | 10 | 7 | 22 |
| Polybutene | 4 | 14 | 16 | — | 4 | 14 | 16 | — | 4 |
| Ozokerite Wax | 4 | 14 | 7 | 23 | 4 | 14 | 7 | 23 | 4 |
| Almond flavor | 0.02 | — | — | — | — | — | — | — | — |
| Blueberry flavor | = | 0.02 | — | — | — | — | — | — | — |
| Cappuccino flavor | — | — | 0.02 | — | — | — | — | — | — |
| Cherry flavor | — | — | — | 0.02 | — | — | — | — | — |
| Coconut flavor | — | — | — | — | 0.02 | — | — | — | — |
| Grape flavor | — | — | — | — | — | 0.02 | — | — | — |
| Milk Chocolate flavor | — | — | — | — | — | — | 0.02 | — | — |
| Orange sorbet flavor | — | — | — | — | — | — | — | 0.02 | — |
| Pineapple flavor | — | — | — | — | — | — | — | — | 0.02 |
| Color Dispersion I | — | — | — | 5 | — | 3 | — | 4 | — |
| Color Dispersion III | — | — | — | = | 2 | — | — | 2 | 7 |
| Color Dispersion IV | 1 | — | 3 | — | 4 | — | 1 | — | — |
| Color Dispersion V | 5 | | 4 | | | | 5 | — | — |
| Color Dispersion VIII | — | — | — | 2 | 1 | — | 2 | — | — |
| Color Dispersion X | — | — | — | — | — | 3 | — | — | — |
| Sorbitan sesquioleate | — | — | — | 0.5 | — | — | 0.5 | — | — |
| Methyl paraben preservative | 0.01 | 0.01 | 0.01 | 0.01 | 0.01 | 0.01 | 0.01 | 0.01 | 0.01 |
| BHA antioxidant | 0.005 | 0.005 | 0.005 | 0.005 | 0.005 | 0.005 | 0.005 | 0.005 | 0.005 |

| Example | 10 | 11 |
|---|---|---|
| Isopropyl palmitate | 50 | — |
| Isopropyl myristate | — | 50 |
| Petrolatum | 22 | 7 |
| Polybutene | 4 | 14 |
| Ozokerite Wax | 4 | 14 |
| Red Raspberry flavor | 0.02 | — |
| Watermelon flavor | = | 0.02 |
| Color Dispersion II | 5 | — |
| Color Dispersion IV | — | 3 |
| Color Dispersion VI | — | 3 |
| Color Dispersion VII | — | 2 |
| Color Dispersion IX | — | 2 |
| Sorbitan sesquioleate | — | 0.5 |
| Methyl paraben preservative | 0.01 | 0.01 |
| BHA antioxidant | 0.005 | 0.005 |

Each of the above compositions is tested for stability by inspecting a sample applied to a person's hand for color intensity when freshly made and again after aging for six months.

The foregoing description is intended as illustrative and is not to be taken as limiting. Still other variations within the spirit and scope of this invention as defined by the claims are possible and will readily present themselves to those skilled in the art.

What is claimed is:

1. An improved lip gloss composition, comprising:
at least one lower alkyl ester of a fatty acid having 12 to 18 carbon atoms, wherein said alkyl ester is a liquid at 25° C.;
an effective amount of a bodying agent having a solubility in said alkyl ester of at least 1 percent by weight at 25° C.;
an effective amount of at least one odorant capable of imparting an odor to said composition;
an effective amount of at least one flavorant capable of imparting a flavor to said composition; and
at least one colorant capable of imparting a color to said composition.

2. An improved lip gloss composition consisting essentially of:
an alky ester of a fatty acid;
an effective amount of a bodying agent, said bodying agent at least partially soluble in said alkyl ester;
at least one odorant dispersed in said composition;
at least one flavorant dispersed in said composition; and
at least one colorant dispersed in said composition.

3. A method of coloring lips, said method comprising the steps of applying to a lip an effective amount of a lip gloss necessary for a user to detect odor, flavor and color of said lip gloss.

4. A method of producing an improved lip gloss composition, said method comprising the steps of:
providing at least one alkyl ester of a fatty acid;
combining an effective amount of bodying agent with said alkyl ester, said bodying agent at least partially soluble in said alkyl ester;
adding at least one odorant to said composition;
adding at least one flavorant to said composition; and
adding at least one colorant to said composition.

5. The composition of claim 1, wherein said alkyl ester is selected from the group consisting of isopropyl myristate and isopropyl palmitate.

6. The composition of claim 1, wherein said bodying agent is selected from the group consisting of ozokerite wax, petrolatum, and polybutene.

7. The composition of claim 2, additionally including a fatty acid ester of a polyhydric alcohol.

8. The composition of claim 2, additionally including a preservative.

9. The composition of claim 2, additionally including an antioxidant.

10. The composition of claim 7, wherein said fatty acid ester is sorbitan sesquioleate.

11. The composition of claim 1, wherein the concentration of said alkyl ester is in the range of 25 to 65 grams per 100 grams of composition.

12. The composition of claim 1, wherein the concentration of said bodying agent is in the range of 15 to 35 grams per 100 grams of composition.

13. The composition of claim 1, wherein the concentration of said odorant is in the range of 0.0005 to 0.5 grams per 100 grams of composition.

14. The composition of claim 1, wherein the concentration of said flavorant is in the range of 0.0005 to 0.5 grams per 100 grams of composition.

15. The composition of claim 1, wherein the concentration of said colorant is in the range of 0.001 to 1 grams per 100 grams of composition.

16. The composition of claim 1, wherein said odorant and said flavorant are identical.

17. The composition of claim 1, wherein said odorant and said flavorant are different.
